(19) United States
(12) Patent Application Publication
(10) Pub. No.: US 2011/0237931 A1
(43) Pub. Date: Sep. 29, 2011

(54) MAGNETIC RESONANCE IMAGING APPARATUS AND SYNCHRONOUS IMAGING METHOD

(76) Inventors: Hideki Kumai, Tokyo (JP); Hiroyuki Itagaki, Tokyo (JP)

(21) Appl. No.: 13/132,163

(22) PCT Filed: Nov. 26, 2009
(86) PCT No.: PCT/JP2009/069945

§ 371 Date: Jun. 1, 2011

(30) Foreign Application Priority Data
Dec. 4, 2008 (JP) 2008-309192

(51) Int. Cl. A61B 5/055

(52) U.S. Cl. 600/413

(57) ABSTRACT

An imaging time is shortened by maintaining a desired image contrast in imaging synchronized with the periodic body movement information regarding the subject. In order to do so, in synchronous measurement of an echo signal synchronized with the trigger information detected from the periodic body movement information regarding the subject with periodic body movement, at least one of a first period before a measurement period of an echo signal and a second period after the measurement period of the echo signal is set, a K space is divided into a plurality of partial regions, and at least one of the first and second periods is set to be different in measurement of an echo signal corresponding to a partial region at a low spatial frequency side and measurement of an echo signal corresponding to a partial region at a high spatial frequency side.

### TECHNICAL FIELD

The present invention relates to a magnetic resonance imaging apparatus and a synchronous measurement method of measuring a nuclear magnetic resonance (hereinafter, referred to as “NMR”) signal from hydrogen, phosphor, or the like in the subject and imaging nuclear density distribution, relaxation time distribution, or the like. In particular, the present invention relates to an improvement in imaging efficiency of synchronous measurement.

### BACKGROUND ART

A nuclear magnetic resonance imaging (hereinafter, referred to as “MRI”) apparatus is an apparatus which measures an NMR signal (echo signal) generated by the nuclear spins, which form tissue of the subject and images the shapes or functions of the head, abdomen, limbs, and the like in a two-dimensional or three-dimensional manner. In the imaging, different phase encoding and different frequency encoding are given to echo signals by the gradient magnetic field, and the echo signals are measured as time series data. The measured echo signals are reconstructed as an image by two-dimensional or three-dimensional Fourier transform.

Since an imaging time of several minutes to several tens of minutes is generally required in the imaging using the MRI apparatus described above, body movement, such as a heartbeat or breathing, of the subject cannot be avoided during the imaging. For this reason, it is known that artifacts caused by body movement appear on an image and this deteriorates the quality of the image.

In the imaging using an MRI apparatus, a method of mounting a cardiac electrode, a pulse wave sensor, or the like on the subject and detecting a biological signal and performing imaging by making a collection timing of an echo signal synchronized with movement of the heart or the like with the detected biological signal as a trigger signal, which is disclosed in Patent Documents 1 to 3, is used as a method for avoiding the above-described problem. That is, in Patent Documents 1 to 3, artifacts on an image caused by body movement can be satisfactorily suppressed by selectively measuring an echo signal in synchronization with the trigger signal only for the phase of relatively small movement of the subject.

### PRIOR ART DOCUMENT

Patent Document 1: JP-A-2008-125986
Patent Document 2: JP-A-2008-136851
Patent Document 3: Japanese Patent No. 4090619

### SUMMARY OF INVENTION

### Technical Problem

In Patent Document 1 or 2, there is a problem in that the imaging time is extended because measurement of an echo signal is selectively performed only for the phase of relatively small movement of the subject. In particular, in the electrocardiographic synchronous measurement method disclosed in Patent Document 3, three-dimensional scanning of repeating an operation, which is for collecting echo signals corresponding to the predetermined amount of slice encoding in a diastolic phase, for each fixed period of a plurality of heartbeats is executed. Accordingly, since an echo signal in the stable blood flow state is measured without being influenced by turbulent blood flow, the contrast of a reconstructed image is improved. However, the problem of extension of an imaging time still remains unsolved as the pulse sequence is repeated every fixed plural heartbeats.

Therefore, it is an object of the present invention to shorten an imaging time by maintaining a desired image contrast in imaging using an MRI apparatus which is synchronized with periodic body movement information regarding the subject.

### Solution to Problem

In order to achieve the above-described object, in the present invention, in synchronous measurement of an echo signal synchronized with the trigger information detected from the periodic body movement information regarding the subject with periodic body movement, at least one of a first period before a measurement period of the echo signal and a second period after the measurement period of the echo signal is set, the K space is divided into a plurality of partial regions, and at least one of the first and second periods is set to be different in measurement of an echo signal corresponding to a partial region at the low spatial frequency side and measurement of an echo signal corresponding to a partial region at the high spatial frequency side.

Specifically, the MRI apparatus of the present invention includes: a detector which detects trigger information from periodic body movement information of a subject; a measurement controller which controls synchronous measurement for measuring an echo signal from the subject in synchronization with the trigger information; and an operation processor which acquires an image of the subject on the basis of K space data in which data of the echo signal is disposed in a K space. In the synchronous measurement, at least one of a first period before a measurement period of the echo signal and a second period after the measurement period of the echo signal is set. The operation processor divides the K space into a plurality of partial regions. The measurement controller sets at least one of the first and second periods to be different in measurement of an echo signal corresponding to a partial region at a low spatial frequency side and measurement of an echo signal corresponding to a partial region at a high spatial frequency side.

In addition, a synchronous imaging method of the present invention includes: a detection step of detecting trigger information from periodic body movement information of a subject; a measurement control step of controlling synchronous measurement for measuring an echo signal from the subject in synchronization with the trigger information; a step of dividing a K space where data of the echo signal is disposed into a plurality of partial regions; and an arithmetic processing step of acquiring an image of the subject using the echo signal. In the synchronous measurement, at least one of a first period before a measurement period of the echo signal and a second period after the measurement period of the echo signal is set. In the measurement control step, at least one of the first and second periods is set to be different in measurement of an echo signal corresponding to a partial region at a low spatial frequency side and measurement of an echo signal corresponding to a partial region at a high spatial frequency side.

### Advantageous Effects of Invention

According to the MRI apparatus and the synchronous imaging method of the present invention, it becomes possible to shorten an imaging time by maintaining a desired image contrast in imaging synchronized with the periodic body movement information regarding the subject.

### BRIEF DESCRIPTION OF DRAWINGS

FIG. 1 is a block diagram showing the overall basic configuration of an example of an MRI apparatus related to the present invention.

FIG. 2 is a view showing an example of division of the three-dimensional K space related to the present invention.

FIG. 3 is a view showing an example of a GUI for division setting of the three-dimensional K space related to the present invention.

FIG. 4 is a view showing another example of the GUI for division setting of the three-dimensional K space related to the present invention.

FIG. 5 is a view showing an example of electrocardiographic synchronous measurement related to a first embodiment of the present invention. FIG. 5A is a view showing an R wave of a cardiac waveform as a trigger signal and a timetable of echo signal measurement. FIG. 5B is a view showing a sequence chart of the pulse sequence for echo signal measurement.

FIG. 6 is a flow chart showing the process flow of the first embodiment of the present invention.

FIG. 7 is a flow chart showing the flow of processing for determining the number of echo data (N) measured for one heartbeat period.

FIG. 8 is a view showing an example of electrocardiographic synchronous measurement related to a second embodiment of the present invention. FIG. 8A shows a trigger signal in the case of acquiring a T2-weighted image and a timetable of echo signal measurement. FIG. 8B is a view showing a sequence chart of the pulse sequence for echo signal measurement.

FIG. 9 is a flow chart showing the process flow of the second embodiment of the present invention.

FIG. 10 is a flow chart showing the flow of processing for setting the number of times of trigger signal waiting (NT) according to a partial region.

### DESCRIPTION OF EMBODIMENTS

Hereinafter, preferred embodiments of an MRI apparatus of the present invention will be described in detail according to the accompanying drawings. In addition, in all drawings for explaining the embodiments of the invention, the same reference numerals are given to those with the same functions and repeated explanation thereof will be omitted.

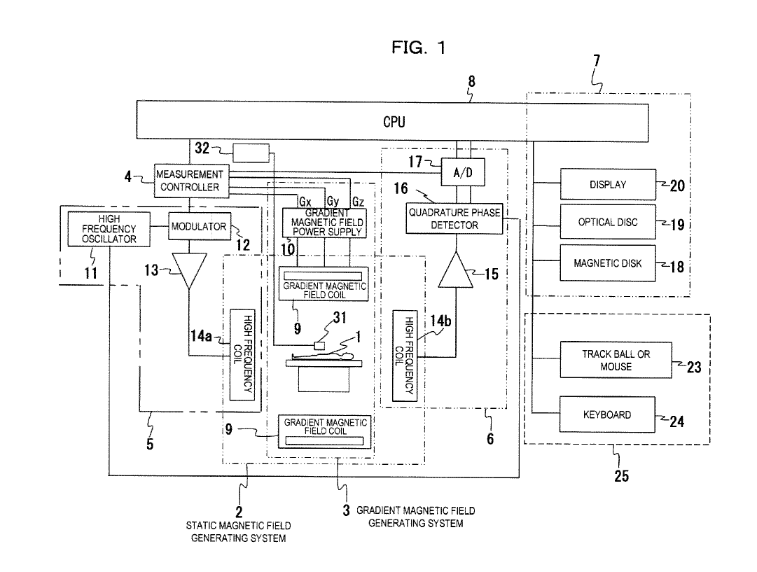

First, the outline of an example of an MRI apparatus related to the present invention will be described on the basis of FIG. 1. FIG. 1 is a block diagram showing the overall configuration of an example of the MRI apparatus related to the present invention. This MRI apparatus acquires a tomographic image of the subject using an NMR phenomenon. As shown in FIG. 1, the MRI apparatus is configured to include a static magnetic field generator 2, a gradient magnetic field generator 3, a signal transmitter 5, a signal receiver 6, an information processor 7, and a measurement controller 4.

The static magnetic field generator 2 generates a uniform static magnetic field in the surrounding space of a subject 1 in a direction perpendicular to the body axis in the case of a vertical magnetic field method and in the body axis direction in the case of a horizontal magnetic field method. A permanent magnet type, a normal conducting type, or a superconducting type static magnetic field generator is disposed around the subject 1.

The gradient magnetic field generator 3 is formed by a gradient magnetic field coil 9 wound in three axial directions of X, Y, and Z, which are a coordinate system (stationary coordinate system) of the MRI apparatus, and a gradient magnetic field power supply 10 which drives each gradient magnetic field coil. The gradient magnetic field power supply 10 of each coil is driven according to a command from the measurement controller 4, which will be described later, so that gradient magnetic fields Gx, Gy, and Gz in the three axial directions of X, Y, and Z are applied to a static magnetic field space where the subject 1 lies. At the time of imaging, a slice-direction gradient magnetic field pulse (Gs) is applied in a direction perpendicular to the slice surface (cross section of imaging) so that a slice surface of the subject 1 is set, and a phase-encoding-direction gradient magnetic field pulse (Gp) and a frequency-encoding-direction gradient magnetic field pulse (Gf) are applied in the two remaining directions, which are perpendicular to the slice surface and are also perpendicular to each other, so that the positional information in each direction is encoded in an echo signal.

The signal transmitter 5 emits a high frequency magnetic field pulse (hereinafter, referred to as an “RF pulse”) to the subject 1 in order to induce an NMR phenomenon in the nuclear spins of the atoms, which form the body tissue of the subject 1, and is configured to include a high frequency oscillator 11, a modulator 12, a high frequency amplifier 13, and a high frequency coil (transmission coil) 14a at the transmission side. A high frequency pulse output from the high frequency oscillator 11 is amplitude-modulated by the modulator 12 at a timing based on the command from the measurement controller 4, and the amplitude-modulated high frequency pulse is amplified by the high frequency amplifier 13 and is then supplied to the high frequency coil 14a disposed adjacent to the subject 1. As a result, an RF pulse is emitted to the subject 1.

The signal receiver 6 detects an echo signal emitted by the NMR phenomenon of the nuclear spins, which form the body tissue of the subject 1, and is configured to include a high frequency coil (receiving coil) 14b at the receiving side, a signal amplifier 15, a quadrature phase detector 16, and an A/D converter 17. An echo signal of a response of the subject 1 induced by the RF pulse emitted from the high frequency coil 14a at the transmission side is detected by the high frequency coil 14b disposed adjacent to the subject 1 and amplified by the signal amplifier 15. Then, at a timing based on the command from the measurement controller 4, it is divided into two signals perpendicular to each other by the quadrature phase detector 16, and each of them is converted into a digital amount by the A/D converter 17 and transmitted to the operation processor 7 as echo data. Hereinafter, the echo signal converted into the digital amount is called echo data.

The measurement controller 4 is control means for controlling the gradient magnetic field generator 3, the signal transmitter 5, and the signal receiver 6 on the basis of a predetermined pulse sequence to repeat the application of an RF pulse and a gradient magnetic field pulse and the measurement of an echo signal. The measurement controller 4 operates under the control of a CPU 8 and transmits various commands, which are required for echo signal collection necessary for reconstruction of a tomographic image of the subject 1, to the gradient magnetic field generator 3, the signal transmitter 5, and the signal receiver 6 in order to control them.

The information processor 7 performs display, saving, and the like of various kinds of data processing and processing results and is configured to include the CPU (operation processor) 8, an external storage device such as an optical disc 19 or a magnetic disk 18, and a display 20. If the echo data from the signal receiver 6 is input to the CPU 8, the echo data is stored in a memory corresponding to the K space in the CPU 8 (hereinafter, description of “arraying an echo signal or echo data in the K space” means that the echo data is written and stored in this memory. In addition, the echo data arrayed in the K space is called K space data). In addition, the CPU 8 executes arithmetic processing, such as signal processing and image reconstruction, on the K space data and displays a tomographic image of the subject 1, which is the result, on the display 20 and also records it in the external storage device.

An operating section 25 receives from an operator an input of various kinds of control information regarding the MRI apparatus or control information regarding the processing performed in the operation processor 7 and is configured to include a track ball or a mouse 23 and a keyboard 24. This operating section 25 is disposed adjacent to the display 20, so that the operator controls various kinds of processing of the MRI apparatus interactively through the operating section 25 while observing the display 20.

The MRI apparatus related to the present invention further includes a cardiac electrode 31, which is mounted on the subject in order to acquire a cardiac waveform signal from the subject, and a cardiac waveform monitor 32, to which a signal from the cardiac electrode is input and which detects the cardiac waveform of the subject and its R wave (trigger signal). The cardiac waveform information (an example of periodic body movement information) detected by the cardiac waveform monitor 32 is input to the CPU 8 through the measurement controller 4, and the measurement controller 4 controls synchronous measurement by controlling each of the above-described sections on the basis of the predetermined pulse sequence in synchronization with a trigger signal (trigger information).

In addition, in FIG. 1, the high frequency coil 14a at the transmission side and the gradient magnetic field coil 9 are provided in the static magnetic field space of the static magnetic field generator 2, into which the subject 1 is inserted, such that they face the subject 1 in the case of a vertical magnetic field method and they surround the subject 1 in the case of a horizontal magnetic field method. In addition, the high frequency coil 14b at the receiving side is provided so as to face or surround the subject 1.

Nuclides imaged by current MRI apparatuses, which are widely used clinically, have a hydrogen nucleus (proton) which is a main constituent material of the subject. The shapes or functions of the head, abdomen, limbs, and the like of the human body are imaged in a two-dimensional or three-dimensional manner by imaging of the spatial distribution of proton density or the information regarding the spatial distribution of a relaxation time of an excited state.

(Division of the K Space)

First, division of the K space related to the present invention will be described. In the MRI apparatus and the synchronous imaging method of the present invention, at least one of a first period before the measurement period of the echo signal and a second period after the measurement period of the echo signal is set in synchronous measurement synchronized with the trigger information detected from the periodic body movement information of the subject. In addition, the K space is divided into a plurality of partial regions, and at least one of the first and second periods is set to be different between measurement of an echo signal corresponding to the partial region at the low spatial frequency side and measurement of an echo signal corresponding to the partial region at the high spatial frequency side.

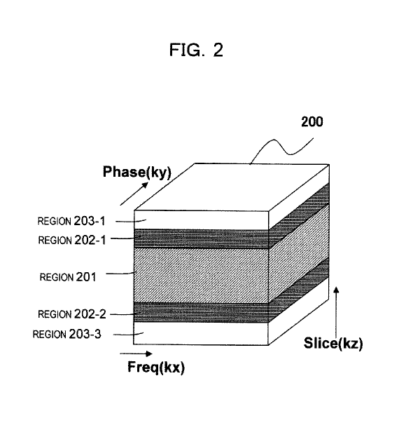

In the case of a two-dimensional K space, the K space is divided in the phase encoding direction. In the case of a three-dimensional K space, the K space is divided in at least one of the slice encoding direction and the phase encoding direction. An example of division of the K space is shown in FIG. 2. FIG. 2 shows an example where each of the positive and negative sides of a three-dimensional K space 200 is divided into three regions (regions 201, 202, and 203) in the slice encoding direction (kz) such that the positive and negative sides are symmetrical with respect to the plane perpendicular to a Slice (kz) axis passing through the origin. Specifically, the CPU 8 divides the three-dimensional K space into the partial region 201 at the low spatial frequency side including the origin and two partial regions 202-1 and 203-1 and two partial regions 202-2 and 203-2 at the high spatial frequency side, which are respectively disposed at both sides of the partial region 201 (regarding the number after a hyphen “-”, 1 means a positive side of the K space slice encoding direction (Kz) and 2 means a negative side thereof). In addition, the rate of division may be changed according to a setting of an operator. FIG. 2 shows an example where the three-dimensional K space is divided such that the partial region 201 is set to 50% of the entire three-dimensional K space and each of the partial regions 202 and 203 is set to 25% of the entire three-dimensional K space. In addition, the K space may also be divided in the phase encoding direction.

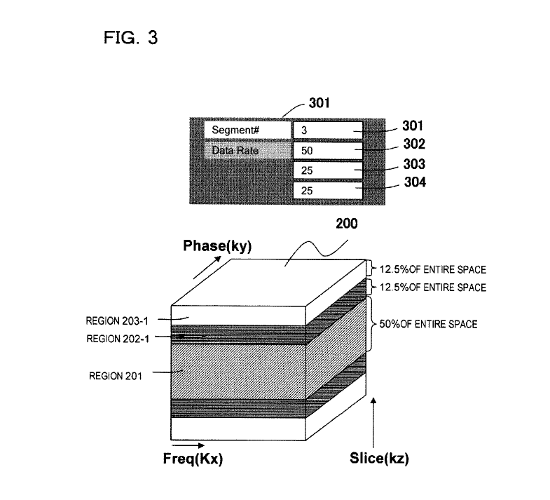

The ratio of each divided region to the entire three-dimensional K space is set as an imaging parameter which can be changed by an operator. For example, an operator may set the ratio of each divided region to the entire three-dimensional K space through a GUI 300 shown in FIG. 3. In the example shown in FIG. 3, an operator inputs and sets the number of divisions Segment# (301) of the three-dimensional K space on the GUI 300, the CPU 8 displays on the display 20 a tag for determining the ratio of each partial region to the entire K space (Data Rate), and the operator inputs and sets a desired numeric value in a rate tag (302, 303, and 304) which is the ratio of each partial region to the entire three-dimensional K space. On the basis of this setting input of the operator, the CPU 8 determines the ratio of each partial region and notifies the measurement controller 4 of the setup information.

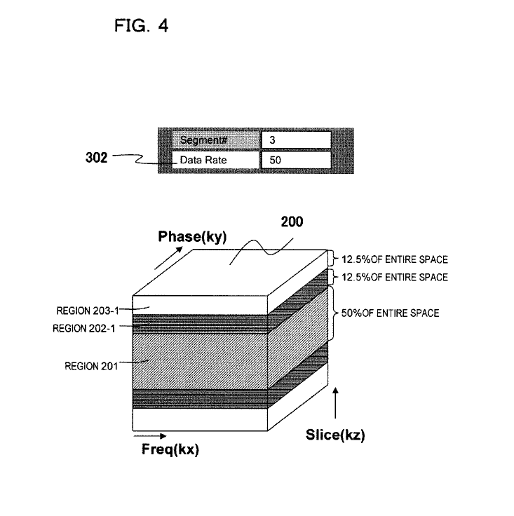

Alternatively, as shown in FIG. 4, an operator may input and set only the number of divisions Segment# 301 of the three-dimensional K space and the ratio Data Rate 302 of a partial region at the low spatial frequency side of the K space to the entire K space, and the CPU 8 may calculate and determine the ratio of each remaining partial region to the entire K space by equally dividing the ratio of the remaining partial regions to the entire K space and notify the measurement controller 4 of the ratio of each partial region.

Hereinafter, each embodiment of the present invention will be described using electrocardiographic synchronous measurement as an example in which the pulsation of the heart and a cardiac waveform are assumed as body movement and periodic body movement information, respectively, and an R wave of a cardiac waveform is set as a trigger signal (an example of trigger information) and measurement of an echo signal is controlled in synchronization with this trigger signal. However, the present invention is not limited to the electrocardiographic synchronous measurement, and may be applied to other periodic body movements, such as the pulsation of a blood flow or periodic movement of a joint.

### First Embodiment

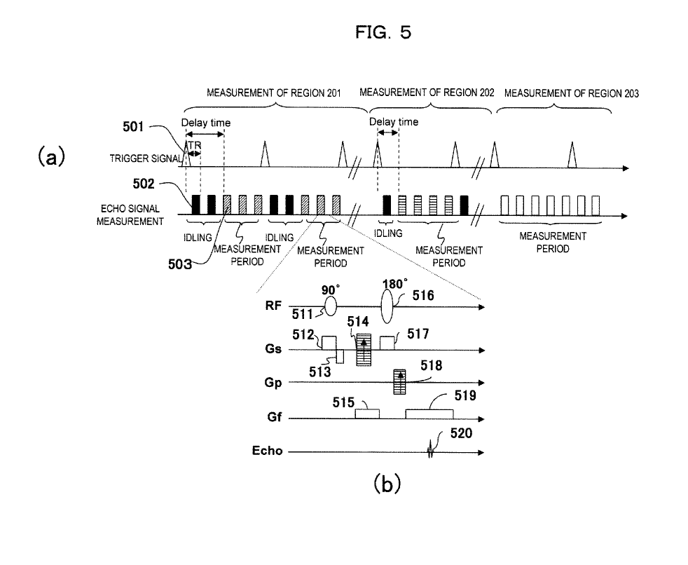

Next, a first embodiment of the MRI apparatus and the synchronous imaging method of the present invention will be described. In the present embodiment, a delay time is set as a first period between the trigger information and a measurement period of an echo signal, and a delay time in the measurement of an echo signal corresponding to a partial region at the high spatial frequency side is set to be shorter than a delay time in the measurement of an echo signal corresponding to a partial region at the low spatial frequency side. The present embodiment is suitable for acquisition of a T1-weighted image, for example. Hereinafter, the present embodiment will be described on the basis of FIGS. 5 to 7.

First, the outline of the present embodiment will be described using FIG. 5. FIG. 5 shows an example in which a cardiac waveform of a subject is detected and electrocardiographic synchronous measurement related to the present embodiment is performed in synchronization with its R wave as a trigger signal. FIG. 5A shows an R wave of a cardiac waveform as a trigger signal and a timetable of echo signal measurement. In addition, FIG. 5B shows a sequence chart of an example of the pulse sequence for echo signal measurement.

When the electrocardiographic synchronous measurement shown in FIG. 5 is started by the operator, the measurement controller 4 drives the pulse sequence in synchronization with a trigger signal 501. In a short repetition period (TR), the measurement controller 4 performs echo signal measurement (503) by repeating this pulse sequence. However, during echo signal measurement in the partial region 201, idling 502 is performed without performing echo signal collection until a fixed time (Delay time) elapses from the trigger signal. This idling 502 is based on the same pulse sequence but an echo signal is not measured, or it is not used for image reconstruction even if it is measured. The reason why idling is performed by setting the fixed Delay time from the trigger signal as described above is to measure an echo signal in a diastolic phase in which movement of the heart is relatively slow since the time band of the Delay time is a systolic phase of the heart in which movement of the heart is fast. This reduces artifacts, which occur on an image due to movement of the heart, so that it becomes possible to acquire a high-resolution image.

After the fixed time (Delay time) elapsed from the trigger signal, the measurement controller 4 repeats the pulse sequence at least once by changing phase encoding in a short repetition time (TR) in a time band, in which body movement of the subject is small, so that an echo signal corresponding to the partial region 201 is measured at least once. Then, the measurement controller 4 measures all echo signals corresponding to the partial region 201 by repeatedly executing echo signal measurement, in which the pulse sequence is repeatedly performed in the short repetition time (TR) after such a Delay time, for a plurality of heartbeats of each heartbeat period.

Then, after proceeding to measurement of an echo signal in the partial region 202, the measurement controller 4 sets the Delay Time short and increases the number of echo signals measured for one heartbeat period. In FIG. 5, the Delay Time at the time of echo signal measurement in the partial region 202 is shorter by one repetition period (1 TR) of the repetition time (TR) of the pulse sequence than that at the time of echo signal measurement in the partial region 201. However, shortening of the Delay Time is not limited either to 1 TR or to the repetition time (TR) unit of the pulse sequence, and may be set arbitrarily.

Similarly, after proceeding to echo signal measurement in the partial region 203, the measurement controller 4 sets the Delay Time at the time of echo signal measurement in the partial region 203 to be shorter than that at the time of echo signal measurement in the partial region 202 and increases the number of echo signals measured for one heartbeat period. The example in FIG. 5 shows a case where the Delay Time is set to 0 (zero). That is, the measurement controller 4 measures an echo signal immediately from the trigger signal without idling.

Generally, an echo signal in the low spatial frequency region is an important signal which has a high strength and controls the quality of an image including the contrast of an image. By reducing artifacts caused by movement by setting the Delay Time so that such an echo signal in the low spatial frequency region can be measured in a diastolic phase in which movement of the heart is relatively slow, an image with a desired contrast can be acquired.

On the other hand, unlike the echo signal in the low spatial frequency region, an echo signal in the high spatial frequency region does not influence the contrast of an image but contributes to the resolution. In addition, since the strength of the echo signal in the high spatial frequency region is also small, the echo signal in the high spatial frequency region has little influence on an image. For this reason, echo signals of the partial regions 202 and 203 are measured in a time band belonging to the systolic phase by reducing the Delay Time similar to the present embodiment, so that the influence on an image is small even if these echo signals are used for image reconstruction. Therefore, measurement may be performed by reducing the Delay Time by an echo signal in the high spatial frequency region similar to the present embodiment. On the contrary, since the number of echo signals which can be measured can be increased by shortening the Delay Time when an echo signal in the high spatial frequency region is measured, a total imaging time can be shortened.

Moreover, in the present embodiment, an echo signal is measured by repeating the pulse sequence in a short repetition time (TR) within one heartbeat period as described above. Accordingly, the present embodiment is suitable for acquisition of a T1-weighted image. Then, as the pulse sequence suitable for acquisition of the T1-weighted image, for example, a spin echo (SE) sequence shown in FIG. 5B is used. RF, Gs, Gp, Gf, and Echo of the sequence chart shown in FIG. 5B indicate an RF pulse, a slice gradient magnetic field, a phase encoding gradient magnetic field, a frequency encoding gradient magnetic field, and an echo signal, respectively. By applying a 90° RF pulse 511 to a subject in a state where a slice selection gradient magnetic field 512 is applied to the subject, magnetization in a desired region is excited by 90° to generate transverse magnetization. Immediately after this, a slice rephase gradient magnetic field 513 is applied so that the phase dispersion of transverse magnetization according to the excitation of a desired region converges again. Then, a slice encoding gradient magnetic field 514 is applied so that the spatial information in the slice encoding direction is encoded in the phase of the echo signal. In addition, by applying a frequency dephase gradient magnetic field 515 in order to generate a symmetrical echo signal, the phase of the transverse magnetization is dispersed. Thereafter, by applying a slice gradient magnetic field 517 and a 180° RF pulse 516, the transverse magnetization is inverted by 180° so as to converge again. Then, the spatial information in the phase encoding direction is encoded in the phase of the echo signal by applying a phase encoding gradient magnetic field 518, and an echo signal 520 is measured while applying a frequency encoding gradient magnetic field 519. Thus, the spatial information in the frequency encoding direction is encoded in the echo signal 520. The echo signal in each partial region is measured by repeating each pulse described above in the short repetition time (TR) while changing at least one of the slice encoding gradient magnetic field 514 and the phase encoding gradient magnetic field 518.

Moreover, in the present embodiment, the strength of an echo signal from the excitation region is stabilized before echo signal measurement by performing idling using the above-described Delay Time. In particular, when measuring an echo signal corresponding to the low spatial frequency region, idling is actively performed using the Delay Time so that an echo signal with a stable strength is measured to acquire a high-resolution image.

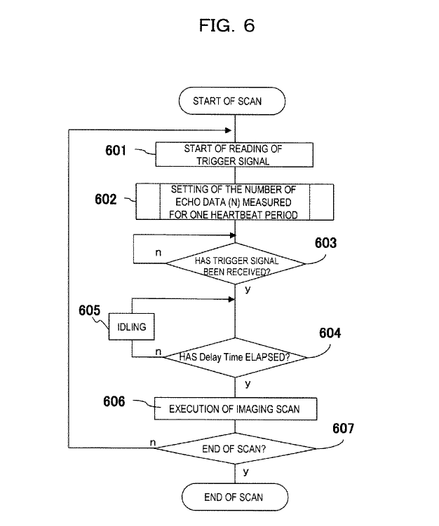

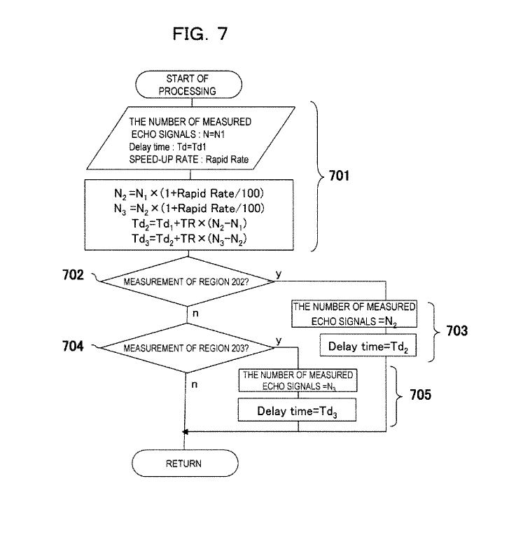

This is the outline of the present embodiment. Next, the operation flow of the present embodiment will be described using flow charts shown in FIGS. 6 and 7. FIG. 6 shows the outline of the operation flow of the present embodiment, and FIG. 7 shows details of processing especially in step 602. These process flows are stored in an external storage device as a program which is executed by the CPU 8 reading the memory and performing execution as necessary. In addition, FIGS. 6 and 7 show the process flow after the operator inputs and sets the number of divisions of the three-dimensional K space in advance through a GUI shown in FIGS. 3 and 4, for example, and the CPU 8 determines the number of divisions of the K space according to the input setup value.

In step 601, when the electrocardiographic synchronous measurement is started by the operator, the measurement controller 4 starts the reading of an R wave in a cardiac waveform of a subject as a trigger signal for performing synchronous measurement.

In step 602, the number of echo signals (N), the Delay time (Td), and the like corresponding to a partial region of the three-dimensional K space, which are measured in one heartbeat period, are determined. Details of this determination processing will be described later on the basis of FIG. 7. In addition, steps 601 and 602 may be executed in reverse order.

In step 603, the measurement controller 4 waits for a trigger signal.

In step 604, when a trigger signal is received, the measurement controller 4 performs wait processing for the Delay time (Td) determined in step 602. During this Delay time (Td), the measurement controller 4 performs idling 605 described above.

In step 606, assuming that a point of time when the Delay time (Td) has elapsed is a timing at which movement of the subject is smallest, echo signal measurement of one heartbeat period shown in FIG. 5 is executed. That is, the measurement controller 4 changes the amount of application of the phase encoding gradient magnetic field or the slice encoding gradient magnetic field and performs measurement corresponding to the number of echo signals measured for one heartbeat period and set in step 602.

In step 607, when echo signal measurement of one heartbeat period ends and electrocardiographic synchronous measurement does not end, the process returns to step 601 to restart reading of the trigger signal, determine the number of echo signals collected during one heartbeat period corresponding to the partial region of the K space, and repeatedly execute steps 601 to 606 until the end of imaging.

This is the outline of the processing flow of the present embodiment. Next, the flow of processing for determining the number of echo signals measured for one heartbeat period in step 602 will be described on the basis of the flowchart shown in FIG. 7.

In step 701, the CPU 8 calculates a Delay Time (Td2) and the number of measured echo signals (N2) at the time of echo signal measurement in the partial region 202 and a Delay Time (Td3) and the number of measured echo signals (N3) at the time of echo signal measurement in the partial region 203 using as references a Delay Time (Td1) and the number of measured echo signals (N1) at the time of echo signal measurement in the partial region 201. In addition, in regard to the number of measured echo signals (N1) and the Delay Time (Td1), for example, the operator may set the number of measured echo signals (N1) and the Delay time (Td1), the operator may set the number of measured echo signals (N1) and then the CPU 8 may calculate the Delay Time (Td1), or the operator may set the Delay time (Td1) and then the CPU 8 may calculate the number of measured echo signals (N1). The Delay Time and the number of measured echo signals in the divided regions 202 and 203 can be calculated by the expressions given below using the imaging speed-up rate Rapid Rate which is set as an imaging parameter by the operator, for example.

N2=N1×(1+Rapid Rate/100), Td2=Td1+TR×(N2−N1) (1)

N3=N2×(1+Rapid Rate/100), Td3=Td2+TR×(N3−N2)

The imaging speed-up rate Rapid Rate may be automatically calculated such that the Delay Time becomes a minimum value eventually, that is, the Delay Time at the time of echo signal measurement in the last partial region (in this case, the partial region 203) becomes a minimum value.

In step 702, the CPU 8 determines whether or not there is a measurement of an echo signal in the partial region 202. If so, the CPU 8 sets the Delay Time and the number of measured echo signals to the values Td2 and N2 of the partial region 202 calculated in Expression (1), respectively, and provides notification thereof to the measurement controller 4 in step 703.

In step 704, the CPU 8 determines whether or not there is a measurement of an echo signal in the partial region 203. If so, the CPU 8 sets the Delay Time and the number of measured echo signals to the values Td3 and N3 of the partial region 203 calculated in Expression (1), respectively, and provides notification thereof to the measurement controller 4 in step 705.

Up until now, the flow of processing for determining the number of echo signals measured for one heartbeat period in step 602 has been described.

As described above, according to the MRI apparatus and the synchronous imaging method of the present embodiment, the number of echo signals measured for one heartbeat period is increased by dividing the three-dimensional K space into a plurality of partial regions in the slice encoding direction and setting the Delay Time when measuring an echo signal differently according to the position of the divided partial region in the slice encoding direction. As a result, the imaging time can be shortened. Specifically, the contrast of a three-dimensional image can be maintained in a desired state by setting the Delay Time when measuring an echo signal in a partial region at the low spatial frequency side such that the desired image contrast is obtained. In addition, since the number of echo signals measured for one heartbeat period in a partial region at the high spatial frequency side is increased by making the Delay Time when measuring an echo signal of the partial region at the high spatial frequency side shorter than the Delay Time when measuring an echo signal of a partial region at the low spatial frequency side, the imaging time can be shortened.

Moreover, in the above explanation of the present embodiment, three-dimensional measurement was exemplified. However, also in two-dimensional electrocardiographic synchronous measurement, the number of echo signals measured for one heartbeat period is increased while maintaining the contrast of a two-dimensional image in a desired state by dividing the two-dimensional K space into a plurality of partial regions in the phase encoding direction and setting the Delay Time when measuring an echo signal differently according to the position of the divided partial region in the phase encoding direction. As a result, the imaging time can be shortened.

### Second Embodiment

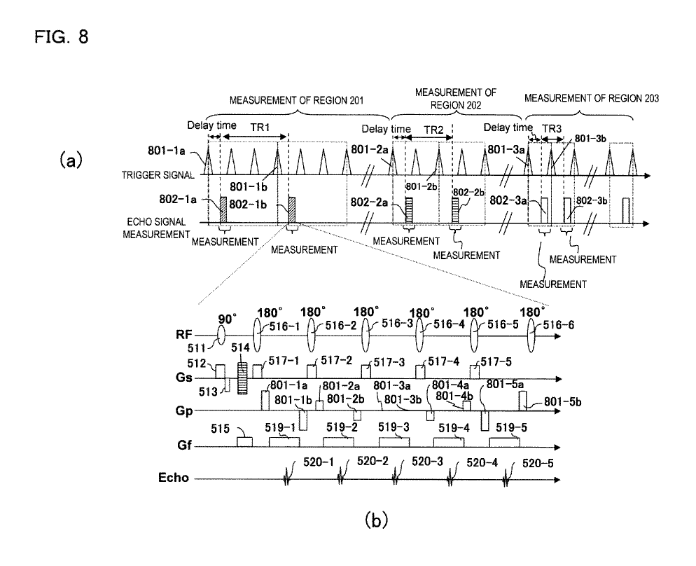

Next, a second embodiment of the MRI apparatus and the synchronous imaging method of the present invention will be described. In the present embodiment, a waiting time from one echo signal measurement period to trigger information which becomes a trigger of the next echo signal measurement period is set as a second period, and a waiting time in the measurement of an echo signal corresponding to a partial region at the high spatial frequency side is set to be shorter than a waiting time in the measurement of an echo signal corresponding to a partial region at the low spatial frequency side. Preferably, the waiting time is set to be different by the integral multiple of one period of periodic body movement. That is, in regard to the repetition time of the pulse sequence for measuring an echo signal, the repetition time in the case of measurement of an echo signal corresponding to the partial region at the high spatial frequency side is set to be shorter than the repetition time in the case of measurement of an echo signal corresponding to the partial region at the low spatial frequency side. The present embodiment is suitable for acquisition of a T2-weighted image, for example. Hereinafter, only points different from the above-described first embodiment will be described on the basis of FIGS. 8 to 10, and explanation regarding the same points will be omitted.

First, the outline of the present embodiment will be described using FIG. 8. FIG. 8 shows an example in which a cardiac waveform of a subject is detected and electrocardiographic synchronous measurement related to the present embodiment is performed in synchronization with its R wave as a trigger signal. FIG. 8A shows a trigger signal in the case of acquiring a T2-weighted image and a timetable of echo signal measurement. FIG. 8B shows a sequence chart of an example of the pulse sequence for echo signal measurement.

The measurement controller 4 starts the pulse sequence after the elapse of a fixed time (Delay time) from detection of a trigger signal 801-1a to perform echo signal measurement 802-1a. A multi-echo type pulse sequence such as an FSE sequence or an EPI sequence, by which a plurality of echo signals can be measured, is used as the pulse sequence, as shown in FIG. 8B. Details thereof will be described later. The measurement controller 4 controls measurement of a predetermined number of echo signals on the basis of such a pulse sequence. After echo signal measurement in this heartbeat period, several (for example, two or three) heartbeats are set as a waiting time in order to obtain the T2-weighted image. After the elapse of this waiting time, the pulse sequence is started again after detection of a trigger signal 801-1b in order to perform measurement 802-1b of an echo signal.

The measurement controller 4 measures an echo signal of the partial region 201 by repeating processing, which has as one unit such measurement of an echo signal after the Delay Time and such waiting during a waiting time of a period of several heartbeats, a plural number of times for each period of a plurality of heartbeats (here, every period of three heartbeats), that is, by setting a three-heartbeat period as a repetition time (TR1) of the pulse sequence.

Then, after proceeding to echo signal measurement in the divided region 202, the measurement controller 4 starts the pulse sequence after the same fixed time (Delay time) as in the measurement in the partial region 201 elapses from detection of a trigger signal 801-2a in order to perform measurement 802-2a of an echo signal. Then, the waiting time is set to be shorter than that at the time of echo signal measurement in the divided region 201. In the example of FIG. 8A, the waiting time after the end of the pulse sequence is shorter by one heartbeat period than that in the case of the divided region 201. In the present embodiment, however, the reduced waiting time is not specified as one heartbeat period and may be set arbitrarily. Moreover, after this waiting time, the pulse sequence is started again after detection of a trigger signal 801-2b in order to perform measurement 802-2b of an echo signal.

The measurement controller 4 measures an echo signal of the partial region 202 by repeating processing, which has as one unit such measurement of an echo signal after the Delay Time and such waiting during a waiting time of a period of one or more heartbeats shorter than that at the time of echo signal measurement in the partial region 201, a plural number of times for each period of a plurality of heartbeats (here, every period of two heartbeats), that is, by setting a two-heartbeat period as a repetition time (TR2) of the pulse sequence.

Similarly, after proceeding to echo signal measurement in the divided region 203, the measurement controller 4 sets the waiting time after pulse sequence operation to be shorter than that at the time of echo signal measurement in the divided region 202 in order to shorten the measurement period of an echo signal. That is, after proceeding to echo signal measurement of the divided region 203, the measurement controller 4 starts the pulse sequence after the same fixed time (Delay time) as in the measurement of the partial region 201 elapses from detection of a trigger signal 801-3a in order to perform measurement 802-3a of an echo signal. Then, the waiting time is set to be shorter than that at the time of echo signal measurement in the divided region 202. In the example of FIG. 8A, the waiting time after the end of the pulse sequence is shorter by one heartbeat period than that in the case of the divided region 202. As a result, the waiting time after the end of the pulse sequence is equal to or shorter than one heartbeat period. Moreover, after this waiting time, the pulse sequence is started again after detection of a trigger signal 801-3b in order to perform measurement 802-3b of an echo signal. That is, the measurement controller 4 measures an echo signal of the partial region 203 with one heartbeat period as a repetition time (TR3) of the pulse sequence.

If the above explanation is summarized, in the present embodiment, the waiting time is set to be different for the repetition time (TR) of the pulse sequence for measuring an echo signal such that the repetition time in the case of measurement of an echo signal corresponding to the partial region at the high spatial frequency side is set to be shorter than the repetition time in the case of measurement of an echo signal corresponding to the partial region at the low spatial frequency side.

In addition, in the measurement of an echo signal corresponding to the partial region at the high spatial frequency side, the Delay time may be set to 0 (zero).

Moreover, in the present embodiment, an echo signal is measured by repeating the multi-echo type pulse sequence for each period of one or more heartbeats according to the partial region as described above. Particularly, in the partial region at the low spatial frequency side, the pulse sequence is repeated for each period of a plurality of heartbeats. Accordingly, the present embodiment is suitable for acquisition of a T2-weighted image. Then, as the pulse sequence suitable for acquisition of the T2-weighted image, for example, an FSE sequence shown in FIG. 8B is used. RF, Gs, Gp, Gf, and Echo of the sequence chart shown in FIG. 8B are the same as those in FIG. 5B. The FSE sequence is a pulse sequence of measuring a plurality of echo signals (520-1 to 520-5) by repeating 180° RF pulses (516-1 to 516-6) of the spin echo sequence, slice gradient magnetic fields (517-1 to 517-5), and frequency encoding gradient magnetic fields (519-1 to 519-5) which are shown in FIG. 5B. Before and after measurement of each echo signal, a pair of phase encoding gradient magnetic fields (801-1a and 801-1b to 801-5a; here, the amount of application of 801-5b and 801-3 is zero) with the same amount of application which has the same absolute amount and different polarities is applied while changing the amount of application for each measurement of an echo signal. For this reason, the phase encoding gradient magnetic field 518 is not applied. Accordingly, it becomes possible to measure a plurality of echo signals (520-1 to 520-5) with different phase encoding using the 90° RF pulse 511 once.

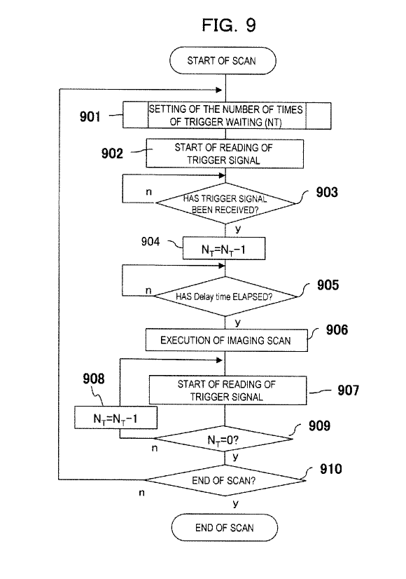

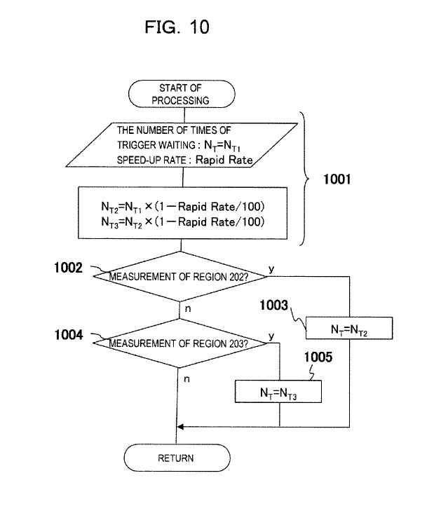

This is the outline of the present embodiment. Next, the operation flow of the present embodiment will be described using flow charts shown in FIGS. 9 and 10. FIG. 9 shows the outline of the operation flow of the present embodiment, and FIG. 10 shows details of processing especially in step 901. These process flows are stored in an external storage device as a program which is executed by the CPU 8 reading the memory and performing execution as necessary. In addition, similarly to the explanation in FIGS. 6 and 7 of the first embodiment described above, it is assumed that an operator inputs and sets the number of divisions of the three-dimensional K space and the CPU 8 determines the number of divisions of the K space according to the input setup value.

In step 901, when the electrocardiographic synchronous measurement is started by the operator, the number of times of trigger signal waiting (NT) is set according to partial regions of the K space where imaging is performed. Details of this setting processing will be described later on the basis of FIG. 10.

In step 902, after the setting of the number of times of trigger signal waiting (NT) described above is completed, the measurement controller 4 starts reading of a trigger signal.

In step 903, the measurement controller 4 receives a trigger signal. Then, in step 904, the measurement controller 4 subtracts 1 from the set number of times of trigger signal waiting (NT).

In step 905, the measurement controller 4 waits for the Delay time (Td) set in advance. If the above-described idling is performed during this Delay Time, the strength of an echo signal measured thereafter is stabilized and a high-resolution image can be obtained accordingly.

In step 906, assuming that a point of time when the Delay time (Td) has elapsed is a timing at which movement of the subject is smallest, the measurement controller 4 executes the pulse sequence shown in FIG. 8B, for example.

In step 907, after the imaging scan ends, the measurement controller 4 reads a trigger signal again. When a trigger signal is received, the measurement controller 4 performs processing step 908 of subtracting 1 from the set number of times of trigger signal waiting (NT). This is executed until NT become 0 (909).

In step 910, if imaging does not end after the above waiting time ends, the process returns to step 901 in which the number of times of trigger signal waiting (NT) is set again and the above-described steps 901 to 909 are repeated until the end of an imaging.

This is the outline of the processing flow of the present embodiment. Next, the outline of the flow of processing for setting the number of times of trigger signal waiting (NT) according to the partial region of the K space in step 901 will be described on the basis of the flow chart shown in FIG. 10.

In step 1001, the CPU 8 sets the number of times of trigger signal waiting (NT2) of the partial region 202 and the number of times of trigger signal waiting (NT3) of the partial region 203 with the number of times of trigger signal waiting (NT1) at the time of measurement in the divided region 201 as a reference. Moreover, in regard to the number of times of trigger signal waiting (NT1), the operator may set the number of times of trigger signal waiting (NT1) or a predetermined value may be set in advance, for example. The number of times of trigger signal waiting in the divided regions 202 and 203 can be calculated by the expressions given below using the imaging speed-up rate Rapid Rate which is set as an imaging parameter by the operator, for example.

NT2=NT1×(1−Rapid Rate/100) (2)

NT3=NT2×(1−Rapid Rate/100)

For example, the imaging speed-up rate Rapid Rate may be automatically calculated such that the Delay Time becomes a minimum value eventually, similar to the first embodiment described above.

In step 1002, the CPU 8 determines whether or not there is a measurement of an echo signal in the partial region 202. If so, the CPU 8 sets the number of times of trigger signal waiting to the value NT2 of the partial region 202 calculated in Expression (2) in step 1003.

In step 1004, the CPU 8 determines whether or not there is a measurement of an echo signal in the partial region 203. If so, the CPU 8 sets the number of times of trigger signal waiting to the value NT2 of the partial region 203 calculated in Expression (2) in step 1005.

Up until now, the flow of processing for setting the number of times of trigger signal waiting (NT) according to the partial region of the K space in step 901 has been described.

As described above, according to the MRI apparatus and the synchronous imaging method of the present embodiment, the imaging time can be shortened by dividing the three-dimensional K space into a plurality of partial regions in the slice encoding direction and setting the waiting time after measuring an echo signal differently according to the position of the divided partial region in the slice encoding direction. Specifically, the contrast of a three-dimensional image can be maintained in a desired state by setting the waiting time when measuring an echo signal in a partial region at the low spatial frequency side such that the desired image contrast is obtained. In addition, the imaging time can be shortened by setting the waiting time when measuring an echo signal in a partial region at the high spatial frequency side to be shorter than the waiting time when measuring an echo signal in a partial region at the low spatial frequency side.

Up until now, each embodiment of electrocardiographic synchronous measurement related to the MRI apparatus and the synchronous imaging method of the present invention has been described. However, the MRI apparatus and the synchronous imaging method of the present invention are not limited to the contents disclosed in each embodiment described above, and other embodiments based on the spirit of the present invention may be adopted. For example, control of the Delay time in each partial region in the first embodiment and control of the waiting time in each partial region in the second embodiment may be executed in combination. In addition, a T2-weighted image may be acquired using the FSE sequence in the first embodiment, or acquisition of a proton-weighted image or the spin echo (SE) sequence may be used in the second embodiment.

### REFERENCE SIGNS LIST

1: subject
2: static magnetic field generating system
3: gradient magnetic field generating system
4: measurement controller
5: signal transmission system
6: signal receiving system
7: signal processing system
8: central processing unit (CPU)
9: gradient magnetic field coil
10: gradient magnetic field power supply
11: high frequency oscillator
12: modulator
13: high frequency amplifier
14a: high frequency coil (transmission coil)
14b: high frequency coil (receiving coil),
15: signal amplifier
16: quadrature phase detector
17: A/D converter
18: magnetic disk
19: optical disc
20: display
21: ROM
22: RAM
23: track ball or mouse
24: keyboard
51: gantry
52: table
53: housing
54: processor

1. A magnetic resonance imaging apparatus comprising:
a detector which detects trigger information from periodic body movement information of a subject;
a measurement controller which controls synchronous measurement for measuring an echo signal from the subject in synchronization with the trigger information; and
an operation processor which acquires an image of the subject on the basis of K space data in which data of the echo signal is disposed in a K space,
wherein in the synchronous measurement, at least one of a first period before a measurement period of the echo signal and a second period after the measurement period of the echo signal is set,
the operation processor divides the K space into a plurality of partial regions, and
the measurement controller sets at least one of the first and second periods to be different in measurement of an echo signal corresponding to a partial region at a low spatial frequency side and measurement of an echo signal corresponding to a partial region at a high spatial frequency side.

2. The magnetic resonance imaging apparatus according to claim 1,
wherein the K space is a three-dimensional space including a slice encoding direction and a phase encoding direction, and division of the K space is performed in the slice encoding direction.

3. The magnetic resonance imaging apparatus according to claim 1,
wherein the measurement controller sets a delay time, as the first period, between the trigger information and the measurement period of the echo signal.

4. The magnetic resonance imaging apparatus according to claim 3,
wherein the measurement controller sets the delay time in measurement of an echo signal corresponding to the partial region at the high spatial frequency side to be shorter than the delay time in measurement of an echo signal corresponding to the partial region at the low spatial frequency side.

5. The magnetic resonance imaging apparatus according to claim 3,
wherein the measurement controller repeats measurement of an echo signal corresponding to the partial region for each one-heartbeat period.

6. The magnetic resonance imaging apparatus according to claim 1,
wherein the measurement controller sets, as the second period, a waiting time from one echo signal measurement period to trigger information which becomes a trigger of the next echo signal measurement period.

7. The magnetic resonance imaging apparatus according to claim 6,
wherein the measurement controller sets the waiting time in measurement of an echo signal corresponding to the partial region at the high spatial frequency side to be shorter than the waiting time in measurement of an echo signal corresponding to the partial region at the low spatial frequency side.

8. The magnetic resonance imaging apparatus according to claim 6,
wherein the measurement controller sets the waiting time to be different by an integral multiple of one period of the periodic body movement.

9. The magnetic resonance imaging apparatus according to claim 6,
wherein the measurement controller repeats measurement of an echo signal corresponding to the partial region at the low spatial frequency side for each period of a plurality of heartbeats.

10. The magnetic resonance imaging apparatus according to claim 6,
wherein the measurement controller sets a delay time, as the first period, between the trigger information and the measurement period of the echo signal, and the delay time is set to be equal regardless of the partial regions.

11. The magnetic resonance imaging apparatus according to claim 1,
wherein for a repetition time of a pulse sequence for measuring the echo signal, the measurement controller sets a repetition time in the case of measurement of an echo signal corresponding to the partial region at the high spatial frequency side to be shorter than a repetition time in the case of measurement of an echo signal corresponding to the partial region at the low spatial frequency side.

12. The magnetic resonance imaging apparatus according to claim 3,
wherein the measurement controller measures the echo signal using a spin echo sequence.

13. The magnetic resonance imaging apparatus according to claim 6,
wherein the measurement controller measures the echo signal using a multi-echo type pulse sequence.

14. The magnetic resonance imaging apparatus according to claim 1, further comprising:
an input setting section which receives a setting of the number of divisions for dividing the K space into a plurality of partial regions and a ratio of at least one partial region to the K space.

15. A synchronous imaging method comprising:
a detection step of detecting trigger information from periodic body movement information of a subject;
a measurement control step of controlling synchronous measurement for measuring an echo signal from the subject in synchronization with the trigger information; and
an arithmetic processing step of acquiring an image of the subject using the echo signal,
wherein in the synchronous measurement, at least one of a first period before a measurement period of the echo signal and a second period after the measurement period of the echo signal is set,
a step of dividing a K space where data of the echo signal is disposed into a plurality of partial regions is further included, and
in the measurement control step, at least one of the first and second periods is set to be different in measurement of an echo signal corresponding to a partial region at a low spatial frequency side and measurement of an echo signal corresponding to a partial region at a high spatial frequency side.

16. The synchronous imaging method according to claim 15,
wherein in the measurement control step, a delay time is set as the first period between the trigger information and the measurement period of the echo signal.

17. The synchronous imaging method according to claim 15,
wherein in the measurement control step, a waiting time from one echo signal measurement period to trigger information which becomes a trigger of the next echo signal measurement period is set as the second period.

18. The synchronous imaging method according to claim 15,
wherein in the measurement control step, for a repetition time of a pulse sequence for measuring the echo signal, a repetition time in the case of measurement of an echo signal corresponding to the partial region at the high spatial frequency side is set to be shorter than a repetition time in the case of measurement of an echo signal corresponding to the partial region at the low spatial frequency side.
